(19) United States
(12) Patent Application Publication
(10) Pub. No.: US 2024/0390619 A1
(43) Pub. Date: Nov. 28, 2024

(54) ADAPTIVE POSITIVE AIRWAY PRESSURE THERAPY SYSTEM

(71) Applicant: Charles William Moyes, III, Austin, TX (US)

(72) Inventor: Charles William Moyes, III, Austin, TX (US)

(21) Appl. No.: 18/675,985

(22) Filed: May 28, 2024

Related U.S. Application Data
(60) Provisional application No. 63/504,482, filed on May 26, 2023.

(51) Int. Cl. A61M 16/00

(52) U.S. Cl. CPC A61M 16/026; A61M 2205/3303; A61M 2205/3355; A61M 2230/04; A61M 2230/10; A61M 2230/205

(57) ABSTRACT

A method of controlling a positive airway pressure (PAP) therapy device involves iteratively performing the actions of receiving at least one biosensor data from at least one biosensor system from a subject utilizing a PAP therapy device during a therapy session. The method receives air pressure reading from a pressure sensor of the PAP therapy device. The method converts the at least one biosensor data into sliding window time-series data. The method packages the sliding window time-series data and air pressure reading into a training set for a classification model to identify sleep quality biomarkers. The method generates an air pressure control through operation of a heuristic model configured by the sleep quality biomarkers. The method adjusts positive airway pressure generated by a blower motor. The method assigns a sleep quality score to the therapy session upon detecting the conclusion of the therapy session and retrains the heuristic model.

### CROSS-REFERENCE TO RELATED APPLICATIONS

This application claims the benefit of U.S. Provisional Patent Application No. 63/504,482 filed May 26, 2023.

### BACKGROUND

Sleep apnea affects millions of individuals worldwide, causing significant health risks and impairing their daily functioning. Conventional treatment methods for sleep apnea often involve positive airway pressure (PAP) machines, which can be intrusive, noisy, and uncomfortable to use while sleeping. These devices require the use of a breathing mask that delivers pressurized air to stent the airway open during sleep. Despite being a relatively non-invasive and highly effective treatment, the compliance rate of individuals that adhere to CPAP therapy is as low as 50%. Many patients seek alternatives that manage their condition in a comfortable and efficient manner.

An objective of the present invention is to provide users with a sleep apnea treatment device that addresses the limitations of existing PAP therapies with a comfortable and efficacious solution. The present invention intends to provide users with an automated system and method that monitors users' sleep quality and continually titrates itself to maintain airway patency, while avoiding uncomfortably high-pressure levels. In order to accomplish this, a preferred embodiment of the present invention comprises a biosensor system, a processing system, and a PAP device.

Furthermore, the system incorporates biological signals to monitor and optimize sleep quality. Thus, the present invention is a PAP medical device that utilizes biological signals and a classification model to improve comfort, efficacy, and compliance.

### BRIEF SUMMARY

### Brief Description of the Several Views of the Drawings

To easily identify the discussion of any particular element or act, the most significant digit or digits in a reference number refer to the figure number in which that element is first introduced.

FIG. 1 illustrates an adaptive positive airway pressure therapy system 100 in accordance with one embodiment.

FIG. 2 illustrates an adaptive positive airway pressure therapy system 200 in accordance with one embodiment.

FIG. 3 illustrates an adaptive positive airway pressure therapy system 300 in accordance with one embodiment.

FIG. 4 illustrates a routine 400 for operating an adaptive positive airway pressure therapy system in accordance with one embodiment.

FIG. 5 illustrates an agent-environment interaction 500 in accordance with one embodiment.

FIG. 6 illustrates a Markov Decision Process 600 in accordance with one embodiment.

FIG. 7 illustrates a recurrent neural network 700 in accordance with one embodiment.

FIG. 8 illustrates a bidirectional recurrent neural network 800 in accordance with one embodiment.

FIG. 9 illustrates a deep bidirectional recurrent neural network 900 in accordance with one embodiment.

FIG. 10A illustrates a long short-term memory 1000 in accordance with one embodiment.

FIG. 10B illustrates features of a LSTM in accordance with one embodiment.

FIG. 11 illustrates an embodiment of a digital apparatus 1100 to implement components and process steps of the system described herein.

FIG. 12 illustrates a diagrammatic representation of a Device 1200 in the form of a computer system within which a set of instructions may be executed for causing the machine to perform any one or more of the functionalities discussed herein, according to an example embodiment.

### DETAILED DESCRIPTION

An adaptive positive airway pressure therapy system is a system and method that incorporates an intelligent control scheme to improve the sleep quality of a subject utilizing a positive airway pressure (PAP) therapy device. The adaptive positive airway pressure therapy system may incorporate an array of biometric sensors to identify sleep quality biomarkers that are then utilized to modify the settings of a PAP therapy device, specifically, controlling the positive airway pressure provided to the subject through their mask. The adaptive positive airway pressure therapy system performs modifications in real-time during a patients sleep therapy session.

In an embodiment, the method of controlling a positive airway pressure (PAP) therapy device involves iteratively performing a set of actions that include receiving at least one biosensor data, comprising electroencephalograph (EEG) data, from at least one biosensor system, comprising an EEG system, operatively coupled to a subject utilizing a PAP therapy device during a therapy session. The method then receives air pressure reading from a pressure sensor of the PAP therapy device. The method then converts the at least one biosensor data into sliding window time-series data in both time and frequency domains through operation of a signal processor. The method then packages the sliding window time-series data and air pressure reading into a training set for a classification model to identify sleep quality biomarkers, through operation of an ingestion engine. The method then generates an air pressure control through operation of a heuristic model configured by the sleep quality biomarkers. The method then adjusts positive airway pressure generated by a blower motor of the PAP therapy device through operation of a controller configured by the air pressure control. When the conclusion of the therapy session is detected, a sleep quality score is assigned to the therapy session through operation of an evaluator. The sleep quality score is then utilized to retrain the heuristic model.

In an embodiment, the at least one biosensor data further comprises electrocardiogram (EKG) data and where the at least one biosensor system further comprises EKG system.

In an embodiment, the at least one biosensor data further comprises electromyogram (EMG) data and where the at least one biosensor system further comprises an EMG system.

In an embodiment, wherein the at least one biosensor system further comprises an oximeter system that generates oxygen saturation data that is provided to the ingestion engine for packaging with the sliding window time-series data and the air pressure reading.

In an embodiment, the at least one biosensor data further comprises electrocardiogram (EKG) data, electromyogram (EMG) data, and oxygen saturation data and where the at least one biosensor system further comprises an EKG system, an EMG system, and an oximeter system.

In an embodiment, the air pressure control adjusts the positive airway pressure generated by the blower motor within a positive airway pressure range.

In an embodiment, a computing apparatus comprises a processor and memory storing instructions that, when executed by the processor, configure the apparatus to perform a set of actions.

The instructions configure the apparatus to iteratively performs a set of actions that include receiving at least one biosensor data, comprising electroencephalograph (EEG) data, from at least one biosensor system, comprising an EEG system, operatively coupled to a subject utilizing a PAP therapy device during a therapy session. The instructions configure the apparatus to then receive air pressure reading from a pressure sensor of the PAP therapy device. The instructions configure the apparatus to then convert the at least one biosensor data into sliding window time-series data in both time and frequency domains through operation of a signal processor. The instructions configure the apparatus to then package the sliding window time-series data and air pressure reading into a training set for a classification model to identify sleep quality biomarkers, through operation of an ingestion engine. The instructions configure the apparatus to then generate an air pressure control through operation of a heuristic model configured by the sleep quality biomarkers. The instructions configure the apparatus to then adjust positive airway pressure generated by a blower motor of the PAP therapy device through operation of a controller configured by the air pressure control. When the conclusion of the therapy session is detected, a sleep quality score is assigned to the therapy session through operation of an evaluator. The sleep quality score is then utilized to retrain the heuristic model.

In an embodiment, the at least one biosensor data further comprises electrocardiogram (EKG) data and where the at least one biosensor system further comprises EKG system.

In an embodiment, wherein the at least one biosensor data further comprises electromyogram (EMG) data and where the at least one biosensor system further comprises an EMG system.

In an embodiment, wherein the at least one biosensor system further comprises an oximeter system that generates oxygen saturation data that is provided to the ingestion engine for packaging with the sliding window time-series data and the air pressure reading.

In an embodiment, the at least one biosensor data further comprises electrocardiogram (EKG) data, electromyogram (EMG) data, and oxygen saturation data and where the at least one biosensor system further comprises an EKG system, an EMG system, and an oximeter system.

In an embodiment, the air pressure control adjusts the positive airway pressure generated by the blower motor within a positive airway pressure range.

In an embodiment, a non-transitory computer-readable storage medium, the computer-readable storage medium including instructions that when executed by a computer, cause the computer to iteratively perform the actions of receiving at least one biosensor data, comprising electroencephalograph (EEG) data, from at least one biosensor system, comprising an EEG system, operatively coupled to a subject utilizing a PAP therapy device during a therapy session. The instructions configure the computer to then receive air pressure reading from a pressure sensor of the PAP therapy device. The instructions configure the computer to then convert the at least one biosensor data into sliding window time-series data in both time and frequency domains through operation of a signal processor. The instructions configure the computer to then package the sliding window time-series data and air pressure reading into a training set for a classification model to identify sleep quality biomarkers, through operation of an ingestion engine. The instructions configure the computer to then generate an air pressure control through operation of a heuristic model configured by the sleep quality biomarkers. The instructions configure the computer to then adjust positive airway pressure generated by a blower motor of the PAP therapy device through operation of a controller configured by the air pressure control. When the conclusion of the therapy session is detected, a sleep quality score is assigned to the therapy session through operation of an evaluator. The sleep quality score is then utilized to retrain the heuristic model.

In an embodiment, the at least one biosensor data further comprises electrocardiogram (EKG) data and where the at least one biosensor system further comprises EKG system.

In an embodiment, wherein the at least one biosensor data further comprises electromyogram (EMG) data and where the at least one biosensor system further comprises an EMG system.

In an embodiment, wherein the at least one biosensor system further comprises an oximeter system that generates oxygen saturation data that is provided to the ingestion engine for packaging with the sliding window time-series data and the air pressure reading.

In an embodiment, the at least one biosensor data further comprises electrocardiogram (EKG) data, electromyogram (EMG) data, and oxygen saturation data and where the at least one biosensor system further comprises an EKG system, an EMG system, and an oximeter system.

In an embodiment, the air pressure control adjusts the positive airway pressure generated by the blower motor within a positive airway pressure range.

The adaptive positive airway pressure therapy device system collects and processes real-time external biological signals from the user via the biosensor system. The biosensor system comprises one or more of the following sensors: an EEG, an EKG, an EMG, a oximeter sensor, accelerometers, and/or gyroscopes—as well as a filter, an amplifier, and analog-to-digital converter (ADC) combined as a signal processor, providing preprocessed digitized sliding window time-series signals in both the time domain and frequency domain.

The EEG is utilized to implicitly model the patient's sleep quality. As a direct model input signal, the patient EEG brainwaves are represented as feature vectors consisting of the time-domain amplitudes and Fourier frequency spectra. These signals are used directly as raw model inputs to implicitly represent the patient's latent sleep quality to anticipate whether additional therapeutic pressure is needed to maintain the patient's breathing, while avoiding increasing pressure in a way that disturbs the patient's sleep.

The EEG system may utilize a variety of different EEG sensors to measure the electrical activity in a subject's brain and capture EEG data. These sensors may include Wet EEG sensors, Dry EEG sensors, wireless EEG sensors, Wearable EEG head bands, Active EEG sensors, and flexible EEG sensors. Wet EEG sensors require a conductive gel to improve contact between the scalp and the sensor, providing high accuracy and sensitivity. However, they can be uncomfortable and cumbersome to set up. In contrast, dry EEG sensors do not require any gel, using small, pin-like electrodes for direct scalp contact. These sensors are favored in various applications, but they tend to be less sensitive than their wet counterparts. Wireless EEG sensors enhance user mobility by eliminating the need for cables. However, these sensors may face issues with interference and data transmission, especially in close. Wearable EEG headbands, integrating multiple sensors into a single device, are designed for everyday use in applications such as basic sleep monitoring. They offer great convenience but generally provide less precise data compared to medical-grade sensors. Active EEG sensors include built-in amplifiers at the sensor site, which boost the EEG signal directly on the scalp, thus enhancing signal quality by reducing potential interference along the electrode cable. Flexible EEG sensors are an emerging technology made from materials that conform more closely to the scalp, potentially improving comfort and signal consistency. These sensors are still largely experimental and aim to merge the benefits of enhanced comfort with reliable data acquisition, promising advances in wearable technology and EEG research.

The EKG time series is designed to model and monitor the patient's heart rate and rhythm for sleep disturbances. For example, the EKG can implicitly determine and track heart arrhythmias or rapid increases in heart rate, indicating arousals during sleep, possibly due to cessations of breath. Conversely, a lower/dipped heart rate would indicate a higher-quality, more restorative form of sleep.

The EKG system may be configured to utilize a variety of EKG sensors to measure the electrical activity of the heart. The traditional EKG sensors are the adhesive electrodes. These electrodes are attached to the skin with a sticky gel that helps conduct the heart's electrical signals. They provide highly accurate readings and are essential for detailed cardiac assessments but can be uncomfortable when worn for extended periods. The suction EKG sensor utilizes suction cups to attach to the skin rather than adhesive. Wearable EKG sensors represent a significant advancement in EKG technology, integrating sensors into devices like chest straps, watches, and even clothing. These sensors are designed for continuous, real-time monitoring and are ideal for patients needing long-term cardiac monitoring, athletes tracking their heart rates during exercise, or individuals engaged in health and fitness programs. While wearable EKGs offer the convenience of mobility and the ability to track heart health over extended periods, they generally do not provide as detailed data as the standard adhesive electrodes. Wireless EKG sensors allow for data transmission to healthcare providers without the need for patients to be physically present in a medical facility. This can be particularly beneficial for patients with mobility issues or those living in remote areas. Wireless EKGs use either a small portable device that connects to a smartphone or computer or a more compact system embedded within a wearable device. In some configurations wireless EKG sensors may be provided by other biometric devices such as EKG capable smart watches.

In an embodiment, an EMG system may utilize a variety of EMG sensors to collect EMG data. The diaphragm EMG is designed to implicitly monitor and predict the patient's breathing rhythms to modulate the frequency (i.e. rise/fall time and period) and phase of the PAP device's output pressure waveform. The EMG predicts and adapts the pressure waveform to the actual breathing of the user by measuring the muscle activations of the diaphragm, which allows for a more comfortable, adaptive breathing waveform in response to anticipated breaths. In an embodiment, the EMG sensors may be surface EMG sensors, these are placed along the lower rib cage or upper abdomen, where the diaphragm is closest to the skin.

In an embodiment, the oximeter device may be a pulse oximeter sensor that measures the patient's blood oxygen levels, including the user's pulse/heart rate, to provide additional signal for sleep quality, as well as detecting and responding to respiratory events. Wearable accelerometers or gyroscopes record patient movements during sleep, another proxy signal for sleep quality and disturbances. The current PAP air pressure is measured by a pressure sensor. It should be further noted that the biosensor system can be created in various ways while still staying within the scope of the adaptive positive airway pressure therapy system. In an additional embodiment, the oximeter data may be collected from non-invasive systems such as Tissue Oxygen Imaging System (TOX-500), Non-invasive Venous Oxygen Saturation (SvO2) Measurement System, and Near-Infrared Spectroscopy (NIRS) Systems.

Tissue Oxygen Imaging System was developed by researchers at the Hefei Institutes of Physical Science of the Chinese Academy of Sciences, this device uses spatial frequency domain spectral imaging to detect tissue oxygen levels over a large area. It projects structured light of different wavelengths onto the tissue and uses the Lambert-Beer law to determine oxygenation by analyzing oxyhemoglobin and deoxyhemoglobin concentrations. This system has shown efficacy in diagnosing conditions like diabetic foot and peripheral vascular disease and provides high accuracy and rapid detection.

Non-invasive Venous Oxygen Saturation Measurement System uses photoplethysmography (PPG) signals with a ring-shaped inflatable air cuff to enhance venous signal detection. By modulating the cuff pressure, the system can differentiate between arterial and venous oxygen signals, providing a non-invasive means to measure venous oxygen saturation. This system allows for continuous monitoring and can be particularly useful for assessing tissue oxygen extraction and perfusion changes.

Continuous NIRS devices are being developed to measure brain oxygenation by placing sensors on the skin to monitor jugular venous oxygen saturation. This technique helps in assessing cerebral perfusion and oxygenation, which is crucial in various medical conditions. This system is non-invasive, provides real-time monitoring, and is useful for brain health assessment and is useful for individuals with decreased peripheral blood flow from conditions such as diabetes.

These components feed into a preprocessing device. Once the input signal passes through the preprocessor, it is converted from an analog signal to a digital signal via the analog-to-digital converter (ADC). The processing system connects to the biosensor system via the ADC. In an embodiment the processing system filters the incoming signals in software, computes model features, and evaluates the machine learning model. The processing system evaluates an ad hoc model or machine learning model in real time to determine the target output pressure (or motor power level) required for the PAP device.

The processing system incorporates the biosensor system and air pressure directly into a tight feedback loop that takes in raw digitized input signal data, preprocesses it, computes model input features, evaluates a machine learning model, and then directly controls the output of the PAP device. This design allows for the pressure to be adjusted dynamically in real-time in response to the current patient's biological state and does not rely on simple heuristics.

In an embodiment, machine learning may be implemented into the processing system via a form of modern deep learning techniques, such as recurrent neural networks (including LSTM), convolutional neural networks, or time-series predictors. The software evaluates the model at a real-time frequency to the order of 10-1000 Hz. To do this, the software repeatedly collects the various signal values, computes the input features, evaluates the model using its pretrained weights, and outputs a target pressure level or motor power level. The model is trained using an objective function over a pre-recorded patient training dataset that uses a weighted sum objective function to minimize the number of sleep disturbance events, minimize the current pressure level, and maximize the quality of the sleep architecture.

In an embodiment, signal PAP device includes hardware components controlled by software or hardware instructions. The PAP device may include software for processing and converting biometric data from the biometric system. The data may then be evaluated and processed through a machine learning system operating on the hardware of the device. In its embodiment, the PAP device is a traditional PAP therapy device that comprises a blower motor controller, a motor power supply, a mask interface, and a pressure sensor. The target pressure can be set and maintained via a PID controller, or the motor power can be an output of the machine learning model itself.

The PAP device is designed to receive various data pertaining to biological signals from the patient to adaptively determine the therapeutic pressure level in real-time. This system allows for the adaptive positive airway pressure therapy system to use machine learning techniques to optimize and minimize the occurrence of sleep-disturbing respiratory events such as apneas, hypopneas, respiratory-effort related arousals, and microarousals. Furthermore, the system uses a machine learning model trained using an objective function which seeks to minimize pressure (while remaining therapeutic), while maximizing the quantifiable sleep quality/sleep architecture of the user.

In one embodiment, the adaptive positive airway pressure therapy system incorporates a wired or wireless electronic device that receives data from various biological sensors that are positioned around the user's body. These signals directly communicate with the processing system to provide inputs for the machine learning model. The PAP device makes use of a blower motor governed by a motor controller that sets the desired target air pressure of the machine to a therapeutic level. With all the components working in tandem with each other, it can be seen that the adaptive positive airway pressure therapy system is a PAP medical device that utilizes additional biological signals to improve comfort, efficiency, and patient compliance.

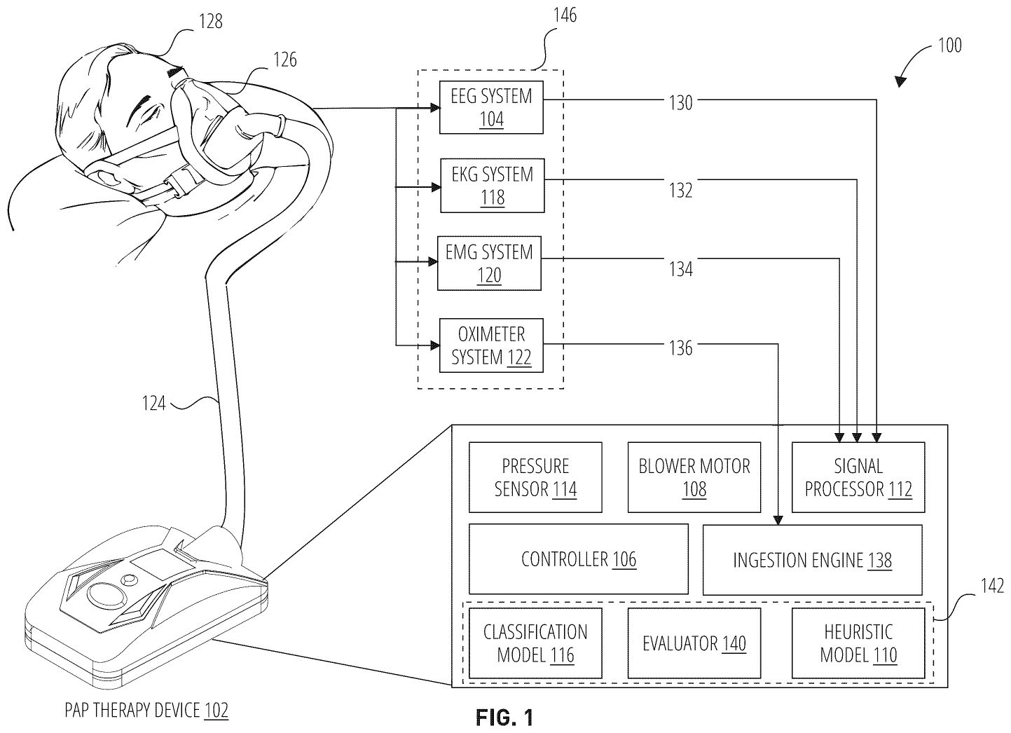

FIG. 1 illustrates an adaptive positive airway pressure therapy system 100 in accordance with one embodiment. The adaptive positive airway pressure therapy system 100 comprises a PAP therapy device 102 in use during a therapy session while a subject 128 is sleeping. The subject 128 is wearing PAP therapy mask 126 that is connected to a hose 124 which provides pressurized air to the subject 128 continuously or adaptively as per the control settings. The PAP therapy device 102 comprises a hose 124, a mask 126, a pressure sensor 114, a blower motor 108, a signal processor 112, a controller 106, an ingestion engine 138, and a machine learning system 142 comprising a classification model 116, an evaluator 140, and a heuristic model 110. The subject 128 is connected to at least one biosensor system 146, comprising the EEG system 104, to monitor the subject 128 sleep quality. These at least one biosensor system 146 may additionally include EKG system 118, an EMG system 120, and an oximeter system 122. Additional biosensor systems may be adapted for use in the system such as an accelerometer and/or gyroscope. The EEG system 104, the EKG system 118, and the EMG system 120 provides EEG data 130, EKG data 132, and EMG data 134, respectively, as biosensor data to the signal processor 112 where the data is filtered, amplified, and processed into sliding window time-series data. The oximeter system 122 provides oxygen saturation data 136 as the biosensor data and is provided to the ingestion engine 138.

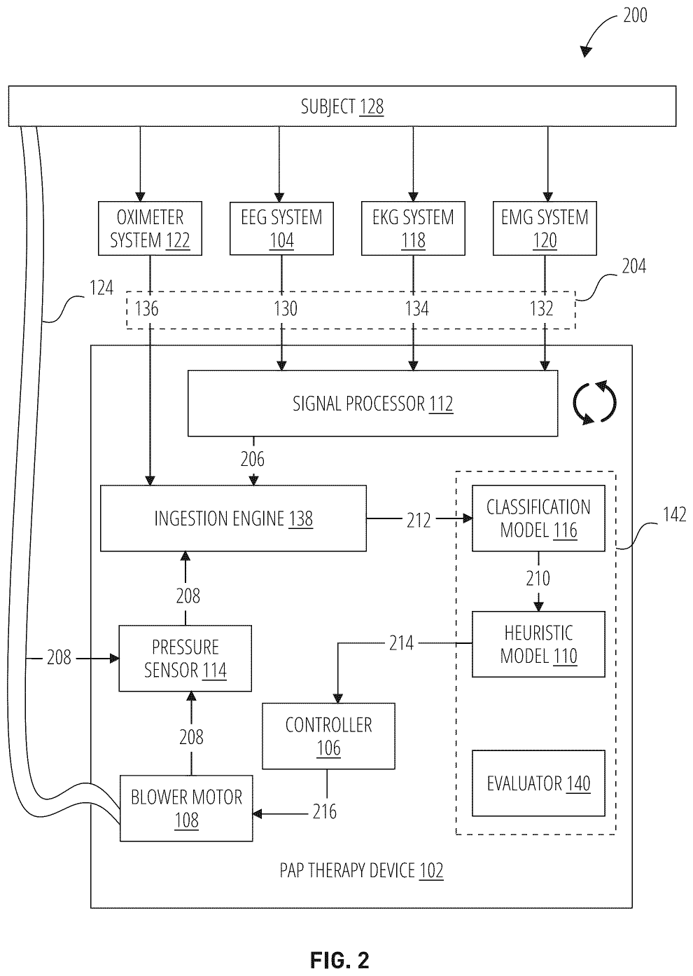

FIG. 2 illustrates an adaptive positive airway pressure therapy system 200 in accordance with one embodiment. The adaptive positive airway pressure therapy system 200 illustrates a PAP therapy device 102 connected to a subject 128 through a hose 124 during a therapy session at night while the subject 128 is sleeping. The subject 128 is operatively coupled to an oximeter system 122, an EEG system 104, an EKG system 118, and an EMG system 120. The EEG system 104, the EKG system 118, and the EMG system 120 communicate at least one biosensor data 204 comprising EEG data 130, EMG data 134, and EKG data 132 to a signal processor 112 throughout the duration of the therapy session. The oximeter system 122 communicates oxygen saturation data 136 to an ingestion engine 138. The PAP therapy device 102 iteratively performs actions during the therapy session to adjust the positive airway pressure 216 provided to the subject 128. The actions performed process the EEG data 130, the EMG data 134, and the EKG data 132 into sliding window time-series data 206 through operation of the signal processor 112. The ingestion engine 138 packages the sliding window time-series data 206, air pressure reading 208 from the pressure sensor 114, and oxygen saturation data 136 from the oximeter system 122 to generate a training set 212 that is provided to the machine learning system 142. The machine learning system 142 comprises a classification model 116, a heuristic model 110, and an evaluator 140. The classification model 116 generates a sleep quality biomarker 210 from the training set 212. The sleep quality biomarkers 210 are utilized to identify the quality of sleep experienced by the subject 128 during the therapy session. The heuristic model 110 utilizes the sleep quality biomarkers 210 to generate an air pressure control 214 to configure the controller 106. The blower motor 108 adjusts the positive airway pressure provided through the hose 124 to the subject 128. In some configurations, the pressure sensor 114 may extrapolate the air pressure reading 208 from the operation of the blower motor 108. In some configurations, the pressure sensor 114 may determine the air pressure from the hose 124 as part of the air pressure reading 208.

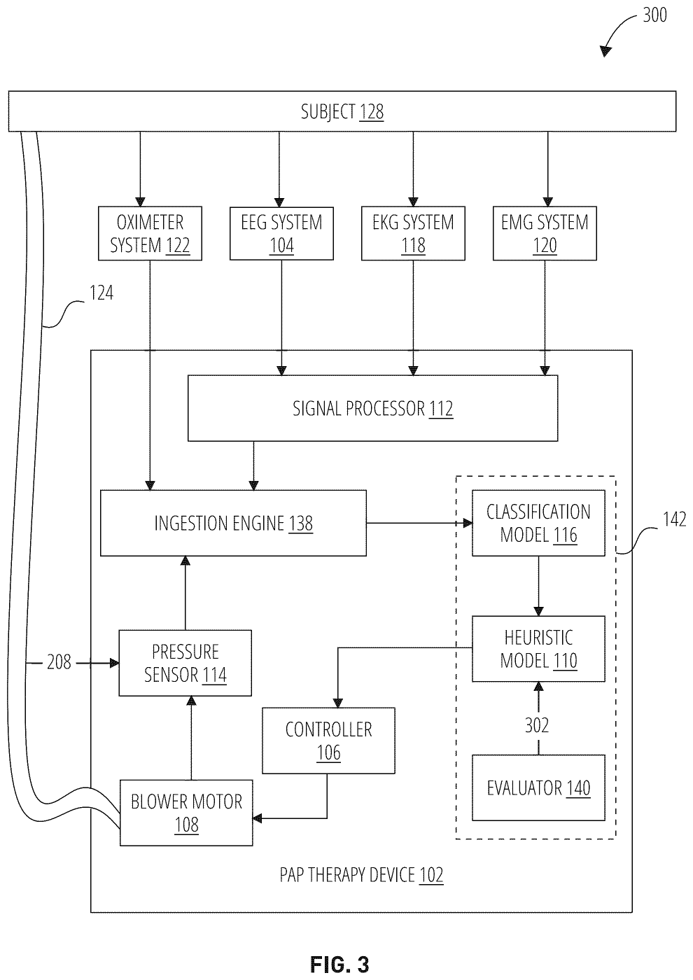

FIG. 3 illustrates an adaptive positive airway pressure therapy system 300 in accordance with one embodiment. In the adaptive positive airway pressure therapy system 300, the therapy session has concluded. This may occur, when the subject 128 awakens and turns off the PAP therapy device 102. In the adaptive positive airway pressure therapy system 300, the oximeter system 122, the EEG system 104, the EKG system 118, and the EMG system 120 may still provide biosensor data to the PAP therapy device 102 but processing by the machine learning system 142 to generate an air pressure control has stopped as the blower motor 108 is no longer providing the positive airway pressure to the subject 128 through the hose 124. The end of a therapy session may be determined after the PAP therapy device 102 is turned off, and not turned back on after a certain period of time. Additionally, the end of a therapy session may be determined by factors such similarities in the time when previous therapy sessions have ended. Upon determining the end of a therapy session, the evaluator 140 may perform an evaluation to generate a sleep quality score 302 for the therapy session. The evaluation may include factors such as the duration of the therapy session, the number of instances that the subject 128 went into deep sleep, how many times the subject 128 was woken up during the therapy session, as well as other factors. In some embodiments, the evaluator may evaluate the objective function to determine the sleep quality score. The sleep quality score is utilized to improve the generation of the air pressure control by the heuristic model 110.

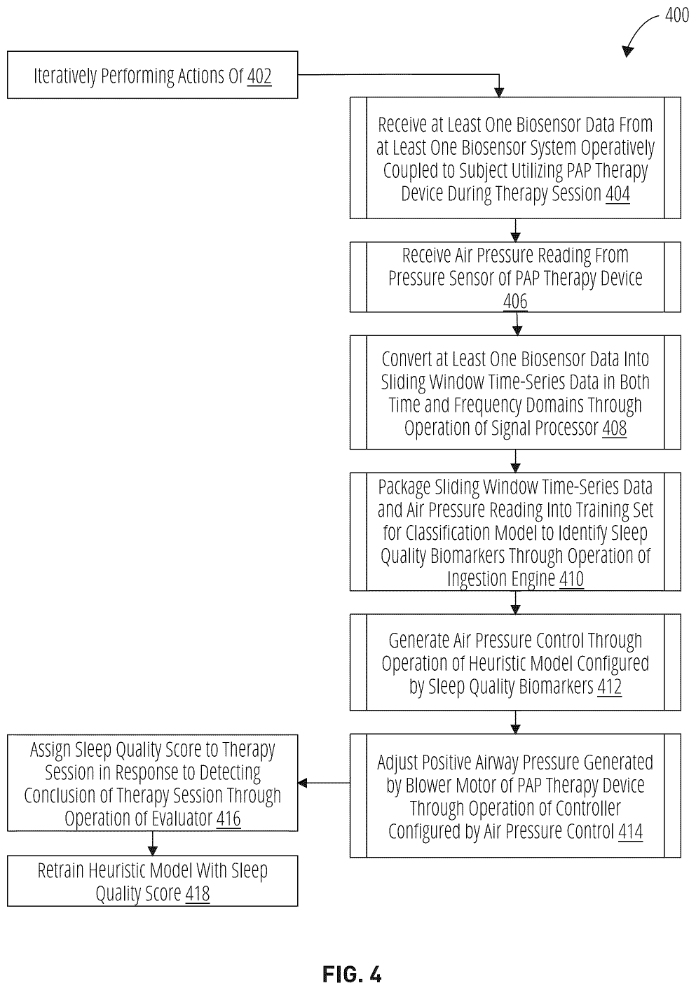

FIG. 4 illustrates a routine 400 for operating an adaptive positive airway pressure therapy system. The routine 400 involves iteratively performing a set of actions during a sleep session (block 402). The actions involve subroutine block 404, subroutine block 406, subroutine block 408, subroutine block 410, subroutine block 412, and subroutine block 414. In subroutine block 404, the routine 400 receives at least one biosensor data, comprising electroencephalograph (EEG) data, from at least one biosensor system, comprising an EEG system, operatively coupled to a subject utilizing a PAP therapy device during a therapy session. In subroutine block 406, the routine 400 receives air pressure reading from a pressure sensor of the PAP therapy device. In subroutine block 408, the routine 400 converts the at least one biosensor data into sliding window time-series data in both time and frequency domains through operation of a signal processor. In subroutine block 410, the routine 400 packages the sliding window time-series data and air pressure reading into a training set for a classification model to identify sleep quality biomarkers, through operation of an ingestion engine. In subroutine block 412, the routine 400 generates an air pressure control through operation of a heuristic model configured by the sleep quality biomarkers. In subroutine block 414, the routine 400 adjusts positive airway pressure generated by a blower motor of the PAP therapy device through operation of a controller configured by the air pressure control. In block 416, the routine 400 assigns a sleep quality score to the therapy session in response to detecting the conclusion of the therapy session through operation of an evaluator. In block 418, the routine 400 retrains the heuristic model with the sleep quality score.

In an embodiment, the machine learning system comprising the classification model, the heuristic model, and the evaluator may be accomplished by a deep reinforcement learning model.

Reinforcement learning (RL) is the area of machine learning that deals with sequential decision-making. In this chapter, we describe how the RL problem can be formalized as an agent that has to make decisions in an environment to optimize a given notion of cumulative rewards. It will become clear that this formalization applies to a wide variety of tasks and captures many essential features of artificial intelligence such as a sense of cause and effect as well as a sense of uncertainty and nondeterminism. This chapter also introduces the different approaches to learning sequential decision-making tasks and how deep RL can be useful.

A key aspect of RL is that an agent learns a good behavior. This means that it modifies or acquires new behaviors and skills incrementally. Another important aspect of RL is that it uses trial-and-error experience (as opposed to e.g., dynamic programming that assumes full knowledge of the environment a priori). Thus, the RL agent does not require complete knowledge or control of the environment; it only needs to be able to interact with the environment and collect information. In an offline setting, the experience is acquired a priori, then it is used as a batch for learning (hence the offline setting is also called batch RL). This is in contrast to the online setting where data becomes available in a sequential order and is used to progressively update the behavior of the agent. In both cases, the core learning algorithms are essentially the same, but the main difference is that in an online setting, the agent can influence how it gathers experience so that it is the most useful for learning. This is an additional challenge mainly because the agent has to deal with the exploration/exploitation dilemma while learning (see § 8.1 for a detailed discussion). But learning in the online setting can also be an advantage since the agent is able to gather information specifically on the most interesting part of the environment. For that reason, even when the environment is fully known, RL approaches may provide the most computationally efficient approach in practice as compared to some dynamic programming methods that would be inefficient due to this lack of specificity.

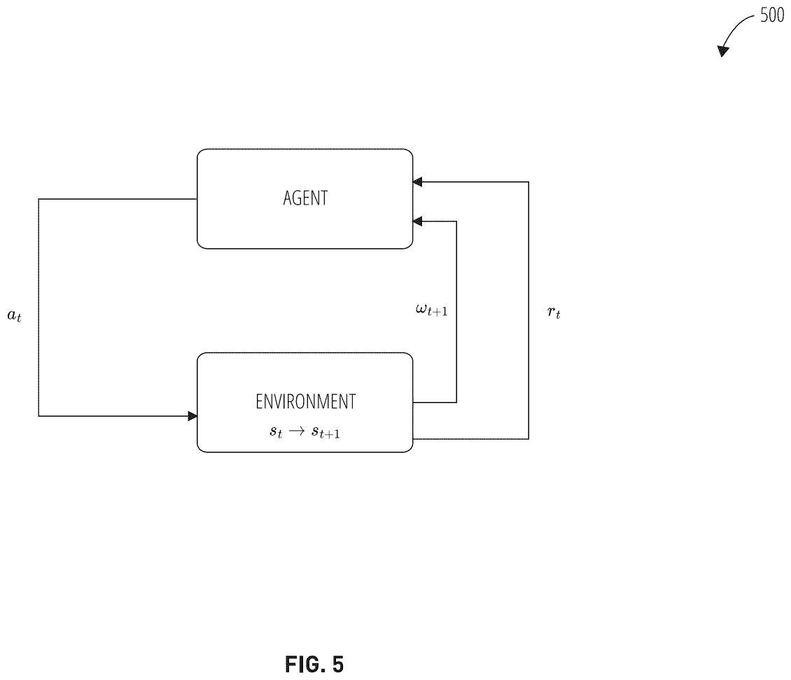

The general RL problem is formalized as a discrete time stochastic control process where an agent interacts with its environment in the following way: the agent starts, in a given state within its environment s₀∈S by gathering an initial observation ω₀∈Ω. At each time step t, the agent has to take an action αₜ∈A. As illustrated in FIG. 5, it follows three consequences: (i) the agent obtains a reward rₜ∈R, (ii) the state transitions to sₜ₊₁∈S and (iii) the agent obtains an observation ωₜ₊₁∈Ω.

A discrete time stochastic control process is Markovian (i.e., it has the Markov property) if:

$$ℙ ( {ω}_{t + 1} \vert {ω}_{t} , {a}_{t} ) = ℙ ( {ω}_{t + 1} \vert {ω}_{t} , {a}_{t} , … , , {ω}_{0} , {a}_{0} ) and \\ ℙ ( {r}_{t} \vert {ω}_{t} , {a}_{t} ) = ℙ ( {r}_{t} \vert {ω}_{t} , {a}_{t} , … , , {ω}_{0} , {a}_{0} ) .$$

The Markov property means that the future of the process only depends on the current observation, and the agent has no interest in looking at the full history.

A Markov Decision Process (MDP) is a discrete time stochastic control process defined as follows:

An MDP is a 5-tuple (S, A, T, R, γ) where:
S is the state space,
A is the action space,
T:S×A×S→[0, 1] is the transition function (set of conditional transition probabilities between states),
R:S×A×S→R is the reward function, where R is a continuous set of possible rewards in a range Rₘₐₓ∈ [symbol] ⁺ (e.g., [0, Rₘₐₓ]),
γ∈[0, 1) is the discount factor.

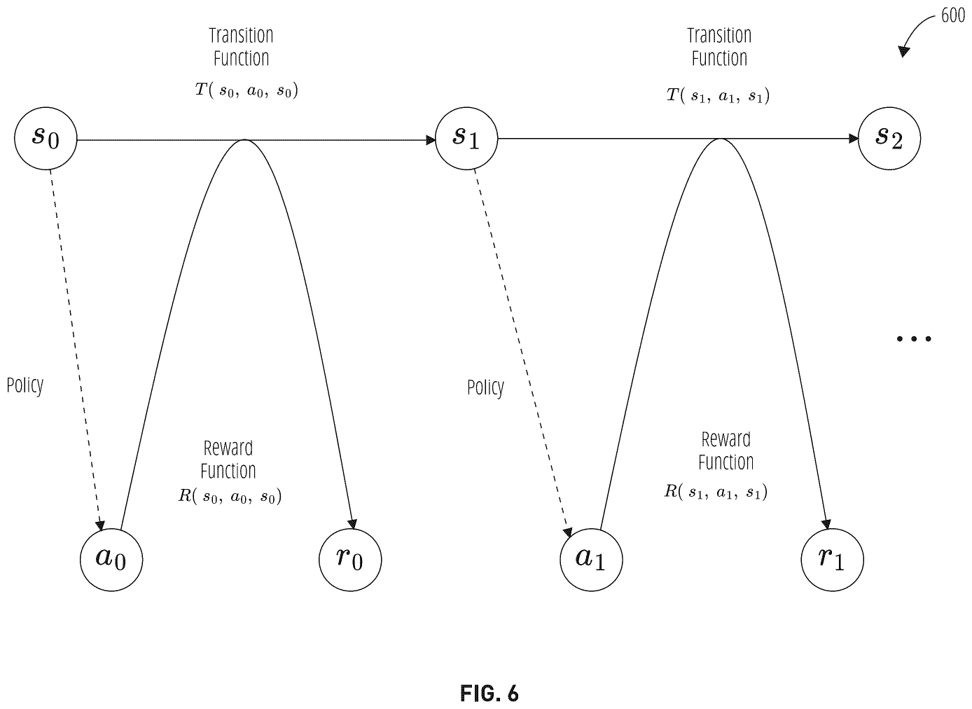

FIG. 6 Illustration of an MDP. At each step, the agent takes an action that changes its state in the environment and provides a reward. The system is fully observable in an MDP, which means that the observation is the same as the state of the environment: ωₜ=sₜ. At each time step t, the probability of moving to sₜ₊₁ is given by the state transition function T (sₜ, aₜ, sₜ₊₁) and the reward is given by a bounded reward function R (sₜ, aₜ, sₜ₊₁)∈R.

A policy defines how an agent selects actions. Policies can be categorized under the criterion of being either stationary or non-stationary. A nonstationary policy depends on the time-step and is useful for the finite-horizon context where the cumulative rewards that the agent seeks to optimize are limited to a finite number of future time steps.

Policies can also be categorized under a second criterion of being either deterministic or stochastic:
In the deterministic case, the policy is described by π(s):S→A.
In the stochastic case, the policy is described by π(s, a):S×A→[0, 1] where π(s, a) denotes the probability that action a may be chosen in state s.

Considering an RL agent whose goal is to find a policy π(s, a)∈Π, as to optimize an expected return of V^{π}(s):S→ [symbol] (also called the V-value function) such that

$${V}^{π} ( s ) = 𝔼 [ {\sum }_{k = 0}^{∞} {γ}^{{k}_{{T}_{t + K}}} \vert {s}_{t} = s , π ] \\ where \\ {r}_{t} = {E}_{a \sim π ( {s}_{t} \cdot )} R ( {s}_{t} , a , {s}_{t + 1} ) , \\ ℙ ( {s}_{t + 1} \vert {s}_{t} , {a}_{t} ) = T ( {s}_{t} , {a}_{t} , {s}_{t + I} ) with {a}_{t} \sim π ( {s}_{t} , \cdot ) ,$$

From the definition of the expected return, the optimal expected return can be defined as:

$${V}^{*} ( s ) = {max}_{π ∈ Π} {V}^{π} ( s ) .$$

In addition to the V-value function, a few other functions of interest can be introduced. The Q-value function Q^{π}(s, a):S×A→ [symbol] is defined as follows:

$${Q}^{π} ( s , a ) = 𝔼 [ {\sum }_{k = 0}^{∞} {γ}^{k} {r}_{t + K} \vert {s}_{t} = s , {a}_{t} = a , π ]$$

This equation can be rewritten recursively in the case of an MDP using Bellman's equation:

$${Q}^{π} ( s , a ) = {\sum }_{s ′ ∈ S ′} T ( s , a , {s}^{′} ) ( R ( s , a , s ′ ) + γ {Q}^{π} ( s ′ , a = π ( s ′ ) ) ) .$$

Similarly to the V-value function, the optimal Q-value function Q*(s, a) can also be defined as:

$${Q}^{*} ( s , a ) = {max}_{π ∈ Π} {Q}^{π} ( s , a )$$

The particularity of the Q-value function as compared to the V-value function is that the optimal policy can be obtained directly from Q*(s, a):

$${π}^{*} ( s ) = {arg max}_{π ∈ A} {Q}^{*} ( s , a )$$

The optimal V-value function V*(s) is the expected discounted reward when in a given state s while following the policy π* thereafter. The optimal Q-value Q*(s, a) is the expected discounted return when in a given state s and for a given action a while following the policy π* thereafter.

It is possible to define the advantage function

$${A}^{π} ( s , a ) = {Q}^{π} ( s , a ) - {V}^{π} ( s ) .$$

This quantity describes how good the action a is, as compared to the expected return when following directly policy π.

Note that one straightforward way to obtain estimates of either V^{π}(s), Q^{π}(s, a), or A^{π}(s, a) is to use Monte Carlo methods, i.e. defining an estimate by performing several simulations from s while following policy π.

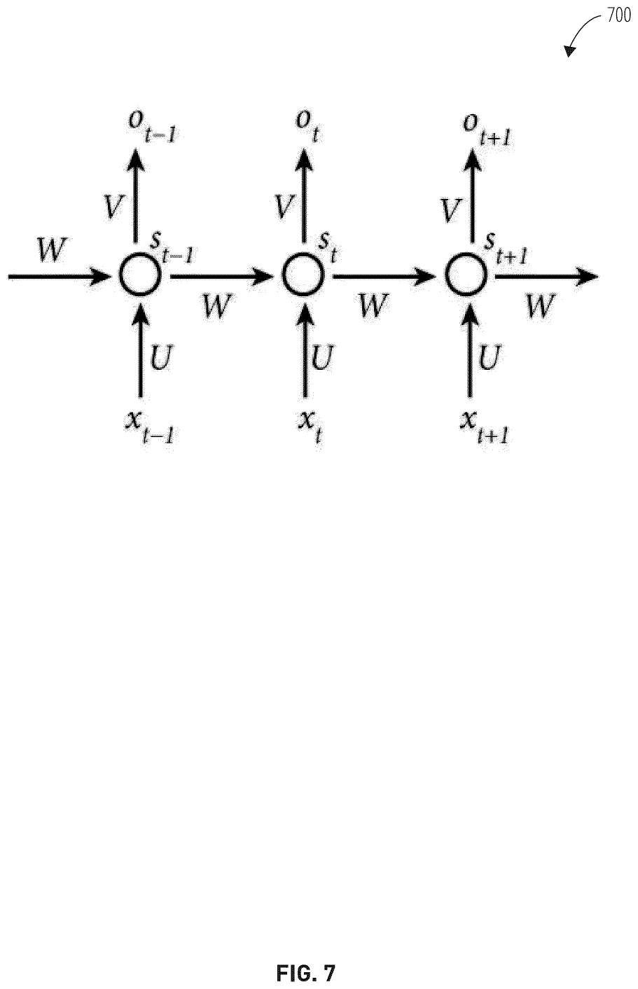

FIG. 7 illustrates a recurrent neural network 700 (RNN). Variable x[t] is the input at stage t. For example, x[1] could be a one-hot vector corresponding to the second word of a sentence. Variable s[t] is the hidden state at stage t. It's the “memory” of the network. The variable s[t] is calculated based on the previous hidden state and the input at the current stage: s[t]=f(Ux[t]+Ws[t−1]). The activation function f usually is a nonlinearity such as tan h or ReLU. The variable s(−1), which is required to calculate the first hidden state, is typically initialized to all zeroes. Variable o[t] is the output at stage t. For example, to predict the next word in a sentence it would be a vector of probabilities across the vocabulary: o[t]=softmax(Vs[t]).

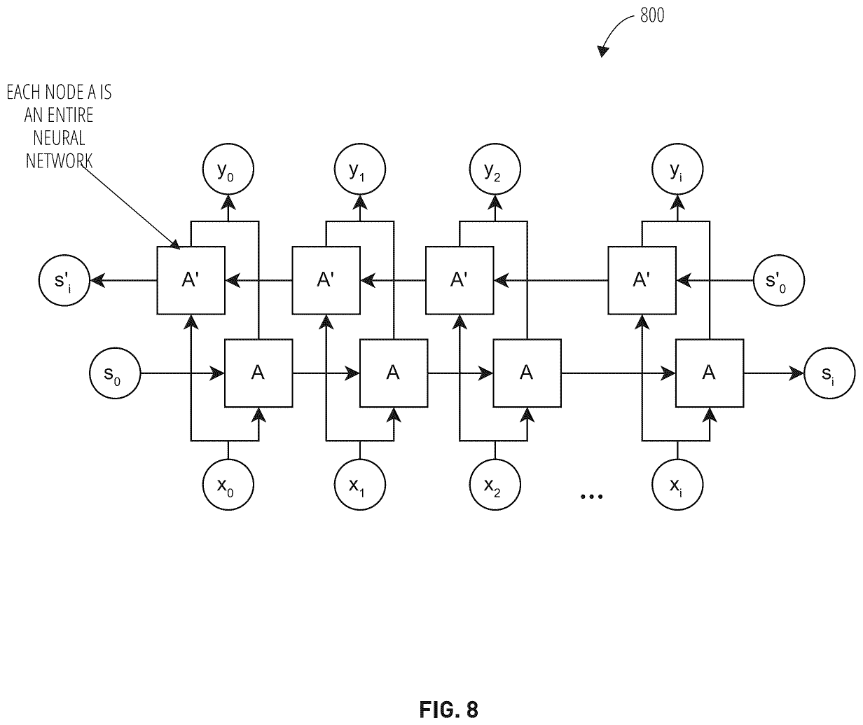

FIG. 8 illustrates a bidirectional recurrent neural network 800 (BRNN). BRNNs are based on the idea that the output at a stage may not only depend on the previous inputs in the sequence, but also future elements. For example, to predict a missing word in a sequence a BRNN will consider both the left and the right context. BRNNs may be implemented as two RNNs in which the output is computed based on the hidden state of both RNNs.

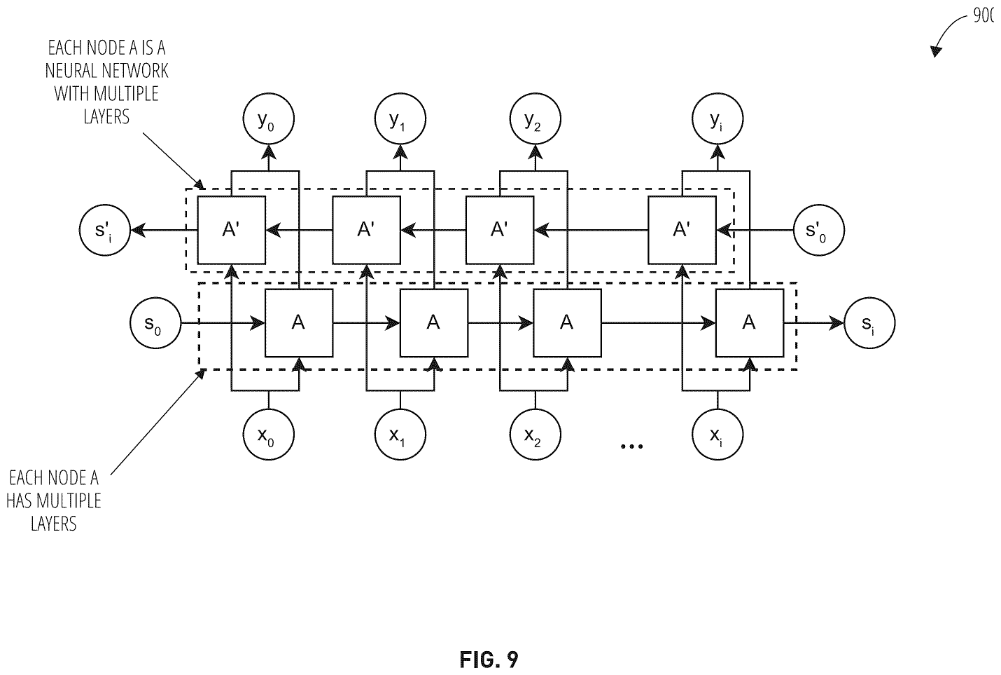

FIG. 9 illustrates a deep bidirectional recurrent neural network 900. Deep BRNNs are similar to BRNNs, but multiple layers per stage. In practice this enables a higher learning capacity but also requires more training data than for single layer networks.

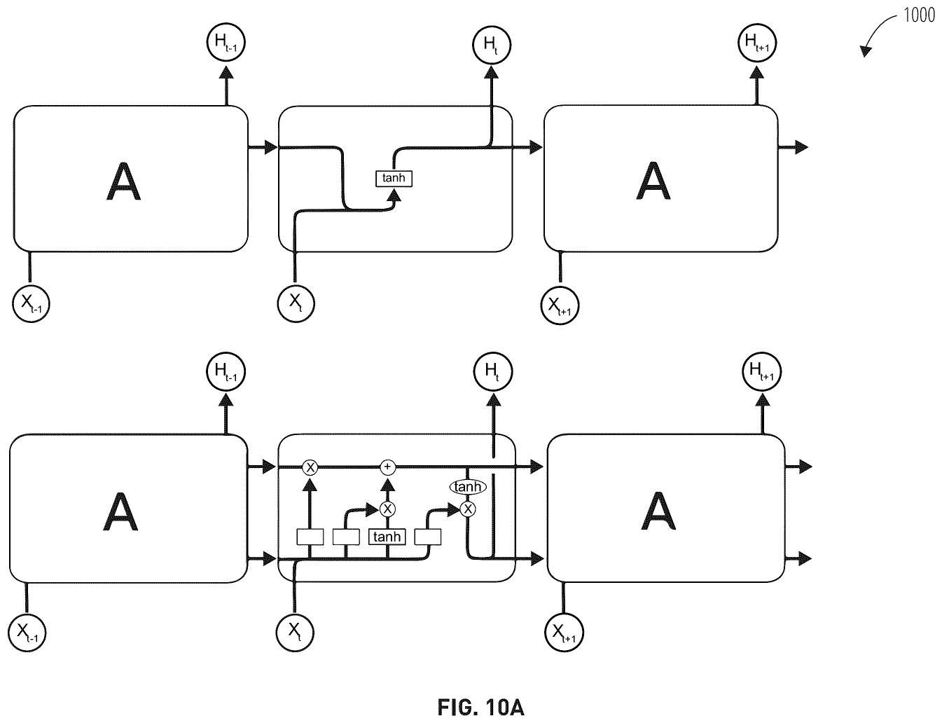

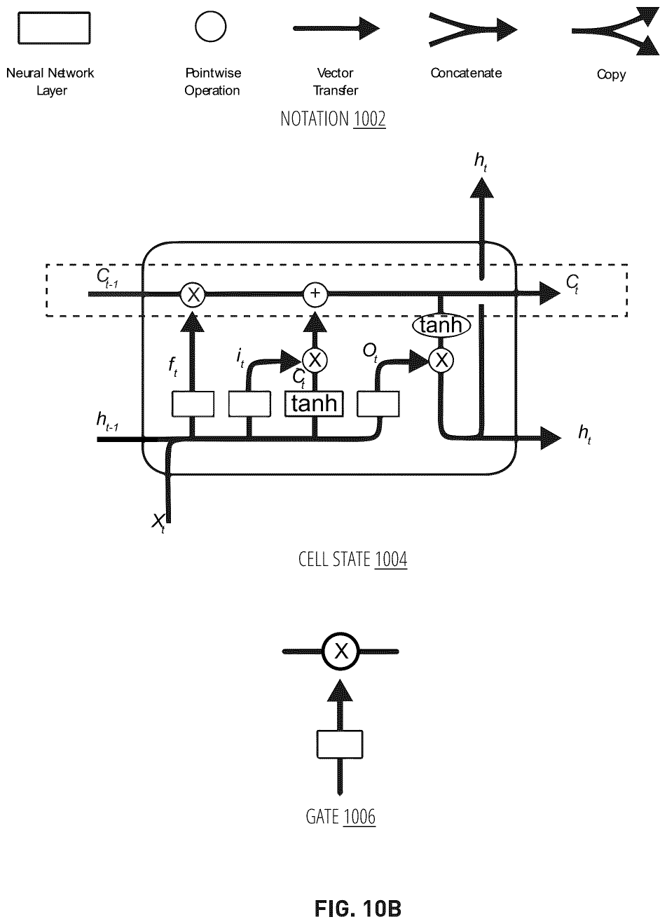

FIG. 10A illustrates an RNN architecture with long short-term memory 1000 (LSTM).

All RNNs have the form of a chain of repeating nodes, each node being a neural network. In standard RNNs, this repeating node will have a structure such as a single tan h layer. This is shown in the upper diagram.

An LSTMs (lower diagram) also has this chain like design, but the repeating node has a different structure than for regular RNNs. Instead of having a single neural network layer, there are typically four, and the layers interact in a particular way.

In an LSTM each path carries an entire vector, from the output of one node to the inputs of others. In FIG. 10B shows notation 1002 for understanding vectors. The circles represent pointwise operations, like vector addition, while the boxes are learned neural network layers. Lines merging denote concatenation, while a line forking denotes values being copied and the copies going to different locations.

An important feature of LSTMs is the cell state Ct, the horizontal line running through the top of the long short-term memory 1000 (lower diagram).

The cell state 1004 is like a conveyor belt. It runs across the entire chain, with only some minor linear interactions. It's entirely possible for signals to flow along it unchanged.

The LSTM has the ability to remove or add information to the cell state, carefully regulated by structures called gates. An example of a gate 1006 is shown in FIG. 10B. Gates are a way to optionally let information through a cell. They are typically formed using a sigmoid neural net layer and a pointwise multiplication operation.

The sigmoid layer outputs numbers between zero and one, describing how much of each component should be let through. A value of zero means “let nothing through,” while a value of one means “let everything through”

An LSTM has three of these sigmoid gates, to protect and control the cell state.

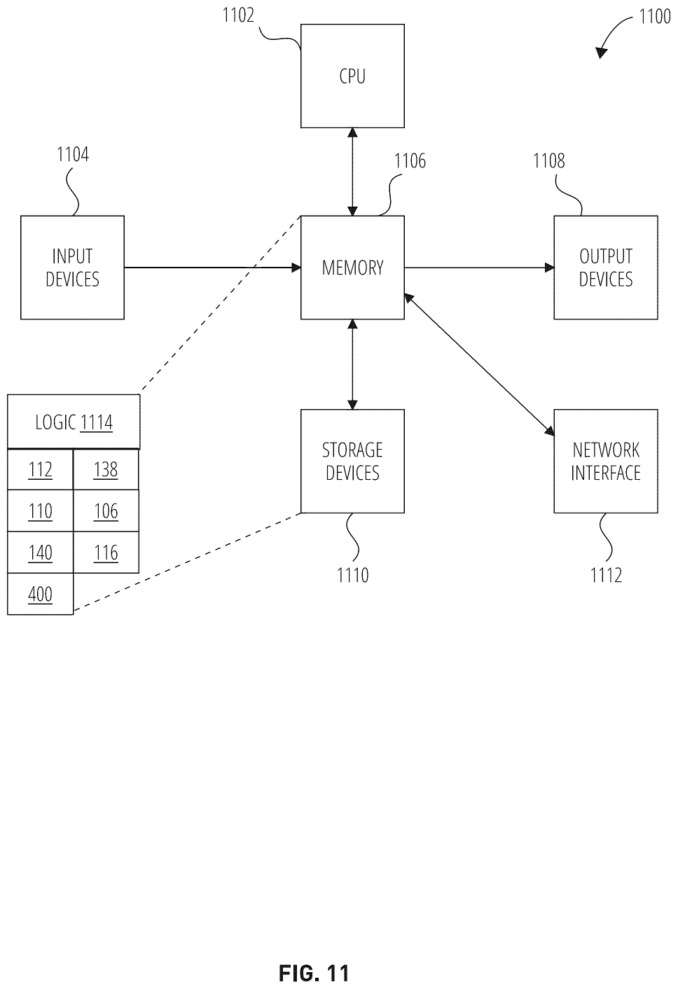

FIG. 11 illustrates an embodiment of a digital apparatus 1100 to implement components and process steps of the system described herein.

Input devices 1104 comprise transducers that convert physical phenomenon into machine internal signals, typically electrical, optical or magnetic signals. Signals may also be wireless in the form of electromagnetic radiation in the radio frequency (RF) range but also potentially in the infrared or optical range. Examples of input devices 1104 are keyboards which respond to touch or physical pressure from an object or proximity of an object to a surface, mice which respond to motion through space or across a plane, microphones which convert vibrations in the medium (typically air) into device signals, scanners which convert optical patterns on two or three dimensional objects into device signals. The signals from the input devices 1104 are provided via various machine signal conductors (e.g., busses or network interfaces) and circuits to memory 1106.

The memory 1106 is typically what is known as a first or second level memory device, providing for storage (via configuration of matter or states of matter) of signals received from the input devices 1104, instructions and information for controlling operation of the CPU 1102, and signals from storage devices 1110.

The memory 1106 and/or the storage devices 1110 may store computer-executable instructions and thus forming logic 1114 that when applied to and executed by the CPU 1102 implement embodiments of the processes disclosed herein. In an embodiment, the memory 1106 and the storage devices 1110 additionally include logic associated with the signal processor 112, the ingestion engine 138, the heuristic model 110, the controller 106, the evaluator 140, the classification model 116, and the routine 400.

Information stored in the memory 1106 is typically directly accessible to the CPU 1102 of the device. Signals input to the device cause the reconfiguration of the internal material/energy state of the memory 1106, creating in essence a new machine configuration, influencing the behavior of the digital apparatus 1100 by affecting the behavior of the CPU 1102 with control signals (instructions) and data provided in conjunction with the control signals.

Second or third level storage devices 1110 may provide a slower but higher capacity machine memory capability. Examples of storage devices 1110 are hard disks, optical disks, large capacity flash memories or other non-volatile memory technologies, and magnetic memories.

The CPU 1102 may cause the configuration of the memory 1106 to be altered by signals in storage devices 1110. In other words, the CPU 1102 may cause data and instructions to be read from storage devices 1110 in the memory 1106 from which may then influence the operations of CPU 1102 as instructions and data signals, and from which it may also be provided to the output devices 1108. The CPU 1102 may alter the content of the memory 1106 by signaling to a machine interface of memory 1106 to alter the internal configuration, and then converted signals to the storage devices 1110 to alter its material internal configuration. In other words, data and instructions may be backed up from memory 1106, which is often volatile, to storage devices 1110, which are often non-volatile.

Output devices 1108 are transducers which convert signals received from the memory 1106 into physical phenomenon such as vibrations in the air, or patterns of light on a machine display, or vibrations (i.e., haptic devices) or patterns of ink or other materials (i.e., printers and 3-D printers).

The network interface 1112 receives signals from the memory 1106 and converts them into electrical, optical, or wireless signals to other machines, typically via a machine network. The network interface 1112 also receives signals from the machine network and converts them into electrical, optical, or wireless signals to the memory 1106.

Terms used herein should be accorded their ordinary meaning in the relevant arts, or the meaning indicated by their use in context, but if an express definition is provided, that meaning controls.

“Circuitry” in this context refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” in this context refers to software logic embodied as processor-executable instructions stored in read-only memories or media.

“Hardware” in this context refers to logic embodied as analog or digital circuitry.

“Logic” in this context refers to machine memory circuits, non transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” in this context refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

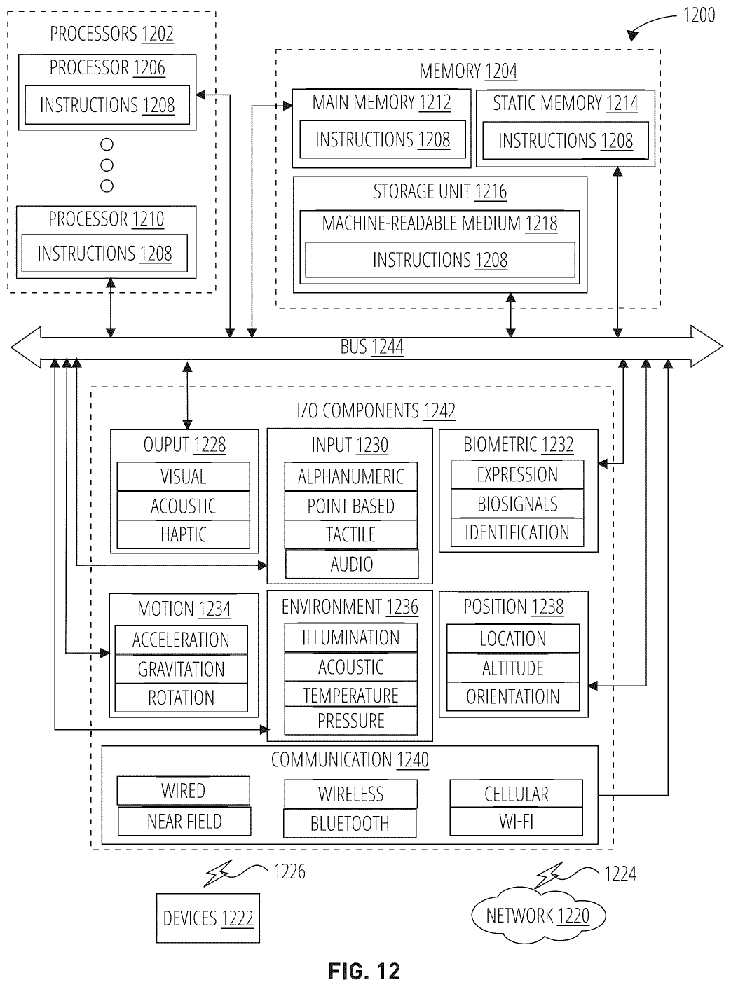

FIG. 12 illustrates a diagrammatic representation of a device 1200 in the form of a computer system within which a set of instructions may be executed for causing the device 1200 to perform any one or more of the methodologies discussed herein, according to an example embodiment. Specifically, FIG. 12 shows a diagrammatic representation of the Device 1200 in the example form of a computer system, within which instructions 1208 (e.g., software, a program, an application, an applet, an app, or other executable code) for causing the Device 1200 to perform any one or more of the methodologies discussed herein may be executed.

The instructions 1208 transform the general, non-programmed Device 1200 into a particular Device 1200 programmed to carry out the described and illustrated functions in the manner described. In alternative embodiments, the Device 1200 operates as a standalone device or may be coupled (e.g., networked) to other machines. In a networked deployment, the Device 1200 may operate in the capacity of a server machine or a client machine in a server-client network environment, or as a peer machine in a peer-to-peer (or distributed) network environment. The Device 1200 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1208, sequentially or otherwise, that specify actions to be taken by the Device 1200.

The Device 1200 may include processors 1202, memory 1204, and I/O components 1242, which may be configured to communicate with each other such as via a bus 1244. In an example embodiment, the processors 1202 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1206 and a processor 1210 that may execute the instructions 1208. The term “processor” is intended to include multi-core processors that may comprise two or more independent processors (sometimes referred to as “cores”) that may execute instructions contemporaneously. Although FIG. 12 shows multiple processors 1202, the Device 1200 may include a single processor with a single core, a single processor with multiple cores (e.g., a multi-core processor), multiple processors with a single core, multiple processors with multiples cores, or any combination thereof.

The memory 1204 may include a main memory 1212, a static memory 1214, and a storage unit 1216, both accessible to the processors 1202 such as via the bus 1244. The main memory 1204, the static memory 1214, and storage unit 1216 store the instructions 1208 embodying any one or more of the methodologies or functions described herein. The instructions 1208 may also reside, completely or partially, within the main memory 1212, within the static memory 1214, within machine-readable medium 1218 within the storage unit 1216, within at least one of the processors 1202 (e.g., within the processor's cache memory), or any suitable combination thereof, during execution thereof by the Device 1200.

The I/O components 1242 may include a wide variety of components to receive input, provide output, produce output, transmit information, exchange information, capture measurements, and so on. The specific I/O components 1242 that are included in a particular machine will depend on the type of machine. For example, portable machines such as mobile phones will likely include a touch input device or other such input mechanisms, while a headless server machine will likely not include such a touch input device. It will be appreciated that the I/O components 1242 may include many other components that are not shown in FIG. 12. The I/O components 1242 are grouped according to functionality merely for simplifying the following discussion and the grouping is in no way limiting. In various example embodiments, the I/O components 1242 may include output components 1228 and input components 1230. The output components 1228 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1230 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1242 may include biometric components 1232, motion components 1234, environmental components 1236, or position components 1238, among a wide array of other components. For example, the biometric components 1232 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1234 may include acceleration sensor components (e.g., accelerometer), gravitation sensor components, rotation sensor components (e.g., gyroscope), and so forth. The environmental components 1236 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1238 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1242 may include communication components 1240 operable to couple the Device 1200 to a network 1220 or devices 1222 via a coupling 1224 and a coupling 1226, respectively. For example, the communication components 1240 may include a network interface component or another suitable device to interface with the network 1220. In further examples, the communication components 1240 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1222 may be another machine or any of a wide variety of peripheral devices (e.g., a peripheral device coupled via a USB).

Moreover, the communication components 1240 may detect identifiers or include components operable to detect identifiers. For example, the communication components 1240 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1240, such as location via Internet Protocol (IP) geolocation, location via Wi-Fi® signal triangulation, location via detecting an NFC beacon signal that may indicate a particular location, and so forth.

### Executable Instructions and Machine Storage Medium

The various memories (i.e., memory 1204, main memory 1212, static memory 1214, and/or memory of the processors 1202) and/or storage unit 1216 may store one or more sets of instructions and data structures (e.g., software) embodying or utilized by any one or more of the methodologies or functions described herein. These instructions (e.g., the instructions 1208), when executed by processors 1202, cause various operations to implement the disclosed embodiments.

What is claimed is:

1. A method of controlling a positive airway pressure (PAP) therapy device comprises:
iteratively performing the actions of:
receiving at least one biosensor data, comprising electroencephalograph (EEG) data, from at least one biosensor system, comprising an EEG system, operatively coupled to a subject utilizing a PAP therapy device during a therapy session;
receiving air pressure reading from a pressure sensor of the PAP therapy device;
converting the at least one biosensor data into sliding window time-series data in both time and frequency domains through operation of a signal processor;
packaging the sliding window time-series data and air pressure reading into a training set for a classification model to identify sleep quality biomarkers, through operation of an ingestion engine;
generating an air pressure control through operation of a heuristic model configured by the sleep quality biomarkers; and
adjusting positive airway pressure generated by a blower motor of the PAP therapy device through operation of a controller configured by the air pressure control;
assigning a sleep quality score to the therapy session in response to detecting the conclusion of the therapy session through operation of an evaluator; and
retraining the heuristic model with the sleep quality score.

2. The method of claim 1, wherein the at least one biosensor system further comprises EKG system and where the at least one biosensor data further comprises electrocardiogram (EKG) data.

3. The method of claim 1, wherein the at least one biosensor system further comprises an EMG system and the at least one biosensor data further comprises electromyogram (EMG) data.

4. The method of claim 1, wherein the at least one biosensor system further comprises an oximeter system that generates oxygen saturation data that is provided to the ingestion engine for packaging with the sliding window time-series data and the air pressure reading.

5. The method of claim 1, wherein the at least one biosensor data further comprises electrocardiogram (EKG) data, electromyogram (EMG) data, and oxygen saturation data and where the at least one biosensor system further comprises an EKG system, an EMG system, and an oximeter system.

6. The method of claim 1, wherein the air pressure control adjusts the positive airway pressure generated by the blower motor within a positive airway pressure range.

7. A computing apparatus comprising:
a processor; and
a memory storing instructions that, when executed by the processor, configure the apparatus to:
iteratively perform the actions of:
receive at least one biosensor data, comprising electroencephalograph (EEG) data, from at least one biosensor system, comprising an EEG system, operatively coupled to a subject utilizing a PAP therapy device during a therapy session;
receive air pressure reading from a pressure sensor of the PAP therapy device;
convert the at least one biosensor data into sliding window time-series data in both time and frequency domains through operation of a signal processor;
package the sliding window time-series data and air pressure reading into a training set for a classification model to identify sleep quality biomarkers, through operation of an ingestion engine;
generate an air pressure control through operation of a heuristic model configured by the sleep quality biomarkers; and
adjust positive airway pressure generated by a blower motor of the PAP therapy device through operation of a controller configured by the air pressure control;
assign a sleep quality score to the therapy session in response to detecting the conclusion of the therapy session through operation of an evaluator; and
retrain the heuristic model with the sleep quality score.

8. The computing apparatus of claim 1, wherein the at least one biosensor data further comprises electrocardiogram (EKG) data and where the at least one biosensor system further comprises EKG system.

9. The computing apparatus of claim 1, wherein the at least one biosensor data further comprises electromyogram (EMG) data and where the at least one biosensor system further comprises an EMG system.

10. The computing apparatus of claim 1, wherein the at least one biosensor system further comprises an oximeter system that generates oxygen saturation data that is provided to the ingestion engine for packaging with the sliding window time-series data and the air pressure reading.

11. The computing apparatus of claim 1, wherein the at least one biosensor data further comprises electrocardiogram (EKG) data, electromyogram (EMG) data, and oxygen saturation data and where the at least one biosensor system further comprises an EKG system, an EMG system, and an oximeter system.

12. The computing apparatus of claim 1, wherein the air pressure control adjusts the positive airway pressure generated by the blower motor within a positive airway pressure range.

13. A non-transitory computer-readable storage medium, the computer-readable storage medium including instructions that when executed by a computer, cause the computer to:
iteratively perform the actions of:
receive at least one biosensor data, comprising electroencephalograph (EEG) data, from at least one biosensor system, comprising an EEG system, operatively coupled to a subject utilizing a PAP therapy device during a therapy session;
receive air pressure reading from a pressure sensor of the PAP therapy device;
convert the at least one biosensor data into sliding window time-series data in both time and frequency domains through operation of a signal processor;
package the sliding window time-series data and air pressure reading into a training set for a classification model to identify sleep quality biomarkers, through operation of an ingestion engine;
generate an air pressure control through operation of a heuristic model configured by the sleep quality biomarkers; and
adjust positive airway pressure generated by a blower motor of the PAP therapy device through operation of a controller configured by the air pressure control;
assign a sleep quality score to the therapy session in response to detecting the conclusion of the therapy session through operation of an evaluator; and
retrain the heuristic model with the sleep quality score.

14. The computer-readable storage medium of claim 1, wherein the at least one biosensor data further comprises electrocardiogram (EKG) data and where the at least one biosensor system further comprises EKG system.

15. The computer-readable storage medium of claim 1, wherein the at least one biosensor data further comprises electromyogram (EMG) data and where the at least one biosensor system further comprises an EMG system.

16. The computer-readable storage medium of claim 1, wherein the at least one biosensor system further comprises an oximeter system that generates oxygen saturation data that is provided to the ingestion engine for packaging with the sliding window time-series data and the air pressure reading.

17. The computer-readable storage medium of claim 1, wherein the at least one biosensor data further comprises electrocardiogram (EKG) data, electromyogram (EMG) data, and oxygen saturation data and where the at least one biosensor system further comprises an EKG system, an EMG system, and an oximeter system.

18. The computer-readable storage medium of claim 1, wherein the air pressure control adjusts the positive airway pressure generated by the blower motor within a positive airway pressure range.
